(19) United States
(12) Patent Application Publication
(10) Pub. No.: US 2005/0214167 A1
(43) Pub. Date: Sep. 29, 2005

(54) High throughput screening with parallel vibrational spectroscopy

(75) Inventors: William B. Archibald, Livermore, CA (US); Alfred W. Archibald, Hillsborough, CA (US)

Correspondence Address: MARTINE PENILLA & GENCARELLA, LLP, 710 LAKEWAY DRIVE, SUITE 200, SUNNYVALE, CA, 94085, US

(73) Assignee: Solus Biosystems, Inc., Palo Alto, CA (US)

(21) Appl. No.: 11/133,490

(22) Filed: May 19, 2005

Related U.S. Application Data
(63) Continuation of application No. PCT/US03/37387, filed on Nov. 21, 2003.
(60) Provisional application No. 60/428,241, filed on Nov. 22, 2002.

(51) Int. Cl. G01N 33/00

(52) U.S. Cl. 422/68.1

(57) ABSTRACT

An apparatus for conducting an analysis of a biological sample is provided. The apparatus includes a sphere designed for application to the biological sample where the sphere is suited for receiving infrared radiation that can be communicated to the biological sample and reflected from the biological sample. The communication to and from the biological sample is through the sphere when the infrared radiation is received.

### CROSS REFERENCE TO RELATED APPLICATION

This application is a continuation and claims 35 U.S.C. § 120 and 365(c) priority from co-pending International Patent Application No. PCT/US2003/037387 filed on Nov. 21, 2003 which designates the United States of America and is entitled “High Throughput Screening with Parallel Vibrational Spectroscopy,” which claims priority from a U.S. Provisional Patent Application No. 60/428,241 filed on Nov. 22, 2002, both of which are incorporated herein by reference in their entirety.

### I. FIELD OF THE INVENTION

The invention relates generally to methods and apparatus for rapid spectrum assay of multiple samples using total internal reflection and related techniques, and in particular to methods and sample holders for optically studying large numbers of chemistries simultaneously.

### II. BACKGROUND OF THE INVENTION

Virtually every area of the biomedical sciences needs to determine the presence, structure, and function of particular analytes that participate in chemical and biological interactions. The needs range from the basic scientific research lab, where biochemical pathways are being mapped and correlated to disease processes, to clinical diagnostics, where patients are routinely monitored for levels of clinically relevant analytes. Other areas include pharmaceutical research, military applications, veterinary, food, and environmental applications. In all of these cases, the presence, quantity, and structure activity relationships of a specific analyte or group of analytes needs to be determined.

Numerous methodologies have been developed to meet this need. The methods include enzyme-linked immunosorbent assays (ELISA), radio-immunoassays (RIA), numerous fluorescence assays, mass spectrometry, calorimetric assays, gel electrophoresis, as well as a host of more specialized assays. Most of the assay techniques require specialized preparations such as chemically attaching a label or purifying and amplifying a sample to be tested. Generally, an interaction between two or more molecules is monitored via a detectable signal relating to the interaction. Typically a label conjugated to either a ligand or anti-ligand of interest generates the signal. Physical or chemical effects produce detectable signals. The signals may include radioactivity, fluorescence, chemiluminescence, phosphorescence, and enzymatic activity. Spectrophotometric, radiometric, or optical tracking methods can be used to detect many labels.

Unfortunately, in many cases it is difficult or even impossible to label one or all of the molecules needed for a particular assay. The presence of a label may interrupt molecular interaction or otherwise make the molecular recognition between two molecules not function for many reasons including steric effects. In addition, none of these labeling approaches can determine the exact nature of the interaction. Active site binding to a receptor, for example, is indistinguishable from non-active site binding, and thus no functional information is obtained from the present detection methodologies. A method to detect interactions that eliminates the need for the label and that yields functional information would greatly improve upon the above mentioned approaches.

Detection technology is commercially very important. The biomedical industry relies on tests for a variety of water-based or fluid-based physiological systems to evaluate protein-protein interactions, drug-protein interactions, small molecule binding, enzymatic reactions, and to evaluate other compounds of interest. Ideally, the technology should not require highly specific probes, such as specific antibodies. The assay should operate by measuring the native properties of molecules and would not require additional label(s) or tracer(s) to detect a binding event. In many applications, the assay should be miniaturizable and handle samples in parallel, so that complex biochemical pathways can be mapped out, or extremely small and numerous quantities of compounds can be used in drug screening protocols. For many applications, the assay should monitor in real time, a complex series of reactions, such that accurate kinetics and structure-activity relationships can be obtained almost immediately.

Vibrational spectroscopy overcomes limitations in this field and is a well established, non-destructive, analytical tool that can reveal much information about molecular interactions. Infrared spectroscopy involves the absorption of electromagnetic radiation generally between 0.770-1000 microns (12,900-10 cm⁻¹), which represent energies on the order of those found in the vibrational transitions of molecular species. Variations in the positions, widths, and strengths of these modes with composition and structure allow identification of molecular species. One advantage of infrared spectroscopy is that virtually any sample, in virtually any state, can be studied without the use of a separate label. Liquids, solutions, pastes, powders, films, fibers, gases, and surfaces can be examined by a judicious choice of sampling techniques. Biological systems such as proteins, peptides, lipids, bio-membranes, carbohydrates, pharmaceuticals, foods, and both plant and animal tissues have been characterized with infrared spectroscopy as reviewed by B. Stuart in Modern Infrared Spectroscopy (Wiley and Sons) Chichester (1996) and in Biological applications of Infrared Spectroscopy (Wiley and Sons) Chichester 1997.

The availability of high-resolution infrared spectrometers has led to time resolved investigations of chemical and biological interactions, which include cell cycle investigations (e.g. H.-Y. Holman, M. C. Martin, E. A. Blakely, K. Bjornstad, W. R. McKinney, Biopolymers (Biospectroscopy) 2000, 57, 329-335), protein-protein interactions (e.g. R. Barbucci, A. Magnani, C. Roncolini, S. Silvestri, Biopolymers 1991, 31, 827-834), polymerization studies (e.g. P. K. Aldridge, J. J. Kelly, J. B. Callis, D. H. Bums, Anal. Chem. 1993, 65, 3581-3585), and solid-phase organic reactions (e.g. B. Yan, J. B. Fell, G. Kumaravel, J. Org. Chem. 1996, 61, 7467-7472). These investigations traditionally have been restricted to one-at-a-time measurements because of single detectors used for conventional infrared spectrometers. Autosamplers have been introduced, which move either the optical path over the samples or a number of samples through the optical path sequentially using a computer controlled system. See for example, the equipment sold by Bruker Inc. of Billerica, Mass. (home page at optics.bruker.com/pages/products/BIO/hts-xt.htm). However, data collection mostly remains serial, making kinetic investigations cumbersome, if not impossible, for a large number of reactions.

Serial one-sample investigations also have been addressed by detector arrays such as a focal plane array which uses infrared spectral imaging for remote sensing, as described by R. Beer Remote sensing by Fourier transform spectrometry (Chemical Analysis v. 120) 1992, Wiley and Sons, New York. Spectral imaging also has been coupled to use of an infrared microscope (See for example U.S. Pat. No. 5,377,003 and references therein and B. Foster, American Laboratory 1997, Feb. 21-29. and P. J. Treado, M. D. Morris, Applied Spectroscopy Reviews 1994, 29(1), 1-38) for imaging studies of plant and animal tissue, polymer dissolution, and polymer liquid crystals. These single sample procedures purport to collect spatially correlated spectral information (i.e. a spectral image). More recently, infrared spectra have been made from multiple samples in parallel and is particularly advantageous for high throughput screening of the large numbers of chemical products in combinatorial investigations. For example, published patent application WO 98/15813 describes the use of parallel detection infrared spectroscopy for monitoring catalytic reactions and other applications of high resolution imagery for “single samples” (see http://www.spectraldimensions.com). This patent application describes measurements primarily in the transmission mode but unfortunately, lacks information needed to make a realistic system. For example, the discussion and figures of sample holders for the transmission measurements do not explain how to transfer samples into a sample array. The assumption presented that a robot would fill sample arrays and then a human would have to “cap” the arrays with an infrared transparent “top” is impractical for an automated high-throughput screening environment. Thus, this described system at best appears limited to the high-resolution imagery on “single-samples.”

Unfortunately, these systems suffer sensitivity and/or speed limitations. One reason is that as sample number increases, the actual size of a signal decreases. The number of photons that can interact with the sample in a short time to generate a meaningful signal decreases dramatically and generally limits both sensitivity and speed. A solution to this problem would open up new areas of discovery and would be particularly important in the burgeoning field of combinatorial chemistry, which require rapid assay of huge numbers of very tiny samples.

### III. SUMMARY

Embodiments of the invention provide a higher throughput analysis of multiple samples and allow real time assay of molecules in their natural environment. Multiple wet samples are analysed via parallel vibrational spectroscopy comprising a source of broadband infrared radiation for probing molecular interactions, a modulator of broadband infrared radiation from the broadband source, a multiple well sample holder having an optical interface with each sample well, wherein the optical interface directs modulated broadband infrared radiation to at least one interface surface between an infrared transparent surface of the sample holder and the sample, allowing internal reflection and subsequent exit of the altered light, an infrared radiation detector for detecting the altered light, and a computer for analyzing data from the infrared radiation detector.

Another embodiment provides a sample holder suitable for simultaneous assay of molecular interactions in multiple wet samples via parallel vibrational spectroscopy, comprising a semiconductor substrate, an array of at least 96 wells for accepting fluid, wherein at least one prismatic feature optically couples to each well and an internal reflection element extending into each well that is optically coupled to the prismatic feature and provides internal reflections within the well. Yet another embodiment provides a method of manufacturing a sample holder for simultaneous assay of molecular interactions in multiple wet samples via parallel vibrational spectroscopy, the holder comprising a semiconductor substrate, an array of wells in the substrate, and at least one internal reflection element extending into each well, the method comprising repeated anisotropic wet etching of the semiconductor substrate to form a two dimensional array of at least 96 wells wherein each prismatic feature has a mean width of between about (e.g. exactly) 5 and about (e.g. exactly) 100 microns and has a mean height of between about (e.g. exactly) 10 and about (e.g. exactly) 10000 microns. In embodiments the mean width may be from about (e.g. exactly) 10 to 75 microns, about (e.g. exactly) 10 to 60 and about (e.g. exactly) 20 to 50 microns. In embodiments the mean height may be between about (e.g. exactly) 25 and about (e.g. exactly) 500 microns, or even between about (e.g. exactly) 50 and about (e.g. exactly) 250 microns.

Yet another embodiment is a sample holder for the simultaneous assay of molecular interactions in multiple wet samples via parallel vibrational spectroscopy, comprising a substrate for holding an array of at least 96 sample wells, and a prismatic structure for each sample well, wherein the prismatic structure comprises a material that is transparent to broadband infrared light of wavelengths between 5 and 10 microns, is at least twice as tall as it is wide and allows the light to enter the optically dense material with an incidence angle that exceeds the critical angle for total internal reflection.

Yet a further embodiment is a tool for detecting effects of chemical compounds on cellular activities or for detecting desirable genetic manipulations in vitro, comprising a source of broadband infrared radiation having wavelengths longer than 5 nanometers, a temperature controlled wet cell sample holder having at least 16 wells that hold and maintain metabolizing cells at a constant temperature, wherein each well has at least one surface in contact with the cells that is transparent to the infrared radiation, one or more prismatic structures for directing the broadband infrared radiation into the infrared radiation transparent surfaces with an incidence angle that exceeds the critical angle for total internal reflection that penetrates a layer of cells in contact with the surface, and an infrared imaging detector that collects reflected light.

Yet another embodiment is a high throughput method for monitoring a reaction involving a set of biomolecules in solution, comprising immobilizing or synthesizing the set of biomolecules on an array surface with different species of biomolecules at discrete immobilizing locations of the array surface, wherein the array surface is transparent to infrared radiation longer than 5 microns wavelength and each immobilizing location is in optical contact with a prismatic structure that directs infrared light with longer than 5 micron wavelength into the array surface with an incidence angle that exceeds the critical angle for total internal reflection that penetrates at least one micron of the solution, irradiating the array surface with broadband infrared radiation of wavelengths longer than 5 nanometers, collecting reflected broadband light spectra from each immobilized location, and calculating multiple absorbance values for the immobilizing locations using Fourier transform.

Yet another embodiment combines multiple infrared sources with parabolic reflectors to generate high intensity light and uses one or more prismatic structures for directing light onto multiple samples.

Yet another embodiment combines visible light and infrared light for simultaneous wide spectral analyses of molecular interactions. Such interactions include short wavelength interactions associated with fluorescence, chemiluminescence and absorbance of chemical moieties of higher energy pi electrons, such as those found in aromatic residues of proteins.

Yet another embodiment enhances signal development by biochemically focusing optical targets to within one half wave period of the probing light, through one or more binding reactions that precede the optical signal development.

Yet another embodiment enhances the performance of drug discovery techniques for chemicals that interact with membrane protein systems, wherein attenuated total internal reflection of biomolecules at cell surfaces is achieved using infrared light with wavelengths that exceed 2 microns and wet samples having intact cells or microsomes immobilized at a probed surface to allow focus on events associated with the membrane proteins.

Yet another embodiment is a method for identifying an individual's propensity to a disease state, or of a disease condition of the individual, comprising the steps of obtaining a spectral fingerprint of a biological specimen of the individual using the instrument described herein; comparing the spectral fingerprint of step a) with a reference indicating a normal spectrum or range of normal spectra to obtain a difference; and comparing the difference with expected differences to make a clinical or predictive conclusion.

Yet another embodiment is a method for detecting a molecular binding event in at least one fluid sample, comprising; exposing the fluid to an electric field gradient in the presence of a pH gradient; irradiating the fluid sample with wide bandwidth, modulated infrared light; detecting infrared radiation obtained by at least transmission, or internal reflectance through the sample, with a broadband infrared radiation detector to generate data; and analyzing the data with a computer to detect the binding event. In yet another embodiment the fluid sample is a protein sample that is exposed to the electric field and pH gradients within a capillary tube.

Yet another embodiment provides an instrument for hyperspectral analysis of isoelectric focusing in real time, comprising; a source of broadband infrared radiation; a modulator of the broadband infrared radiation; a fluid sample holder that allows infrared light produced from the source of broadband infrared radiation to enter; an infrared radiation detector for detecting the infrared light that exits the fluid sample holding chamber; and a computer for analyzing data from the infrared radiation detector. The broadband infrared radiation light may pass through the sample holder in a transmission mode. The broadband infrared radiation light may undergo internal reflection prior to exit of the reflected light. The fluid sample holder may have a 1 dimensional capillary. The fluid sample holder may be in a multiple sample titer plate format and the samples may be exposed to air. Furthermore, at least two samples may be tested simultaneously within two different pH ranges. Also, the infrared radiation detector may be a focal plane array detector.

Another embodiment provides a transfer mechanism selected from the group consisting of a programmable transfer fluidic mechanism for transferring at least one sample at a time from a standard titer plate into the fluid sample holder, and a programmable transfer fluidic mechanism for transferring at least one sample at a time from the fluid sample holder into a mass spectrometer.

Another embodiment provides a sample holder for the simultaneous assay of molecular interactions in multiple wet samples via parallel vibrational spectroscopy, comprising a substrate for holding an array of at least 3 sample units, wherein each unit comprises: a capillary fed sample well having at least one surface that is infrared transparent; at least one sample injection port; at least one sample removal port; and capillaries that connect each port to the sample well. Still further, the at least one transparent region of each sample well may comprise one or more infrared transparent materials selected from the group consisting of an alkali halide salt, CaF2, BaF2, ZnSe, Ge, Si, thin polyethylene, AMTIR and KRS-5. Yet further, the sample holder sample array may be microfabricated using lithography and standard semiconductor processing techniques. The sample holder may further comprise an infrared opaque substance selected from the group consisting of a plastic, glass, wax, polymer, metal, or elastomer, and may comprise at least 96 sample units.

In yet another embodiment an apparatus for conducting an analysis of a biological sample is provided. The apparatus includes a sphere designed for application to the biological sample where the sphere is suited for receiving infrared radiation that can be communicated to the biological sample and reflected from the biological sample. Communication to and from the biological sample is through the sphere when the infrared radiation is received.

In another embodiment, a method to analyze a biological sample with a sphere is provided. The method includes applying a sphere to a solution including the biological sample and attracting the biological sample to the sphere. The method further includes attaching the sphere to a conduit and applying infrared radiation through the conduit and the sphere to the biological sample. The method also includes transmitting infrared radiation reflected back from the biological sample through the sphere and the conduit to an infrared radiation detector.

In yet another embodiment, an apparatus for conducting an analysis of a biological sample, the apparatus is provided which includes a plurality of fibers configured to collect spectral information from multiple biological samples and infrared optics coupled to the plurality of fibers where the infrared optics is configured to pass light into the plurality of fibers and receive light from the plurality of fibers. The apparatus also includes a detection system configured to simultaneously measure the light received from the plurality of fibers.

In another embodiment, a method to analyze a biological sample with a sphere is provided which includes applying the biological sample to the sphere. The method further includes applying infrared radiation through the sphere and to the biological sample. The method also includes receiving reflected infrared radiation from the sphere for analysis of the biological sample.

In yet another embodiment, an apparatus for conducting an analysis of a biological sample is provided which includes an object designed for application to the biological sample. The object is suited for receiving infrared radiation that can be communicated to the biological sample and reflected from the biological sample. The communication to and from the biological sample is through the object when the infrared radiation is received.

### IV. BRIEF DESCRIPTION OF THE FIGURES

FIG. 1 shows a schematic outline of optics used for reflectance measurements of attenuated total reflection according to an embodiment.

FIG. 2 is a schematic diagram of an imaging spectrometer for transmission measurements according to an embodiment of the invention.

FIG. 3 shows representative sample holders for transmission measurements according to embodiments of the invention.

FIG. 4 shows a prismatic device for attenuated total reflection according to an embodiment.

FIG. 5 shows a hemispheric surface for attenuated total reflection according to an embodiment.

FIG. 6 shows an attenuated total reflection sampling assay according to an embodiment.

FIG. 7 shows a multiple internal reflection sampling assay according to an embodiment.

FIGS. 8a and 8b show the combination of multiple infrared sources with a mirror to increase the amount of parallel probing light according to an embodiment.

FIG. 9 shows a biospecific capture layer for parallel attenuated total reflectance according to an embodiment.

FIG. 10 shows a 64 well micro array prepared by wet etching silicon and adding sample wells.

FIG. 11 shows an etch mask used for photolithography to make a 64 well micro array.

FIG. 12 is a cross sectional view of a 64 well micro array prepared by wet etching silicon.

FIG. 13 shows an embodiment of a spherical proximity assay.

FIG. 14 shows a representative fiber optic sampling device according to an embodiment.

FIG. 15 shows multiple fiber optic sampling devices used together with a common hyperspectral detection system.

FIG. 16 shows the use of simultaneous hyperspectral imaging with isoelectric focusing separation.

### V. DETAILED DESCRIPTION

The inventors studied the problem of multiple sample spectroscopy with a total system viewpoint and realized that the quantity of light processed per sample is a major limitation to the assay of many small samples simultaneously. That is, the spectroscopic analysis of a large number of samples in parallel requires a much higher flow of total light to obtain parallel information for each sample simultaneously. This system obstacle was addressed by: i) increasing the amount of starting light with parabolic optics and multiple light sources; ii) adopting a high bandwidth system that uses wide spectrum light and Fourier analysis, allowing much higher light fluxes and consequent information flow; iii) discovery of prismatic structures and alternative sample formats that greatly increase light throughput while permitting large sample numbers; iv) discovery of miniature sample holder designs that can be mass produced by semiconductor processing techniques; and v) discovery of biochemical and cellular focusing techniques that further optimize signal energy use for improved signal to noise. Each of these discoveries contributes to improved performance, singly and in combination, and facilitates the use higher sample number spectroscopic assays, as further detailed below.

Wide Bandwidth Systems for Multiple Sample Assays

Embodiments of the invention utilize light spectra of multiple wavelengths to measure absorption and/or transmission spectra from arrays of multiple samples simultaneously. In contrast to many previous techniques, the high bandwidth systems of embodiments of the present invention use entire spectral regions, combined with Fourier analysis, for much greater total light usage and real time detection of individual wavelengths without requiring narrow light filtering. Most other spectroscopic systems discard the vast majority of light from a light source via bandpass filtering or by use of a diffraction grating and selection of a wavelength. The high bandwidth and Fourier analysis are particularly desirable in combination with prismatic structures and small sized but high sample number assay targets.

Fourier transform methods used in embodiments of the invention are known and have been used for spectroscopy and for total internal reflectance as exemplified in U.S. Pat. No. 5,416,325 issued to Buontempo et al., May 16, 1995. The contents of this patent, and particularly the described methods for maximizing the ratio of signal to noise for low light intensity signals specifically are incorporated by reference in their entireties. The contents of U.S. Pat. No. 5,777,736 issued to Horton on Jul. 7, 1998; U.S. Pat. No. 5,254,858 issued to Wolfman et al. on Oct. 19, 1993; U.S. Pat. No. 4,382,656 issued to Gilby on May 10, 1983; U.S. Pat. No. 4,240,692 issued to Winston on Dec. 23, 1980; U.S. Pat. No. 4,130,107 issued to Rabl et al. on Dec. 19, 1978; and U.S. Pat. No. 5,361,160 issued to Normandin et al. on Nov. 1, 1994 also provide details for use of Fourier transform spectroscopic methods are particularly incorporated by reference, and represent art known to the skilled artisan.

Light from a light source is modulated and an interferometer for this purpose preferably is used within a light passageway having focusing and/or beam steering optics to manage the light beam. The managed beam contacts (by reflection or transmission) each sample simultaneously and then is directed toward the detector, which preferably is a two dimensional detector. The detector collects data simultaneously from the samples and transfers the data to a computer for storage and processing.

The interferometer may be placed on the source side to interrupt the probing light before contact with sample or it may be on the detector side to interrupt the light between the sample and the detector. In either embodiment the interferometer modulates the light prior to detection by the detector. For embodiments that utilize infrared light, as much of the beam path as possible should be in a controlled environment to limit error due to water absorption. It is highly desirable to control the amount of water vapor and carbon dioxide in the environment surrounding the sample to achieve a stable baseline. Drift in the temperature, humidity, or chemical content of the medium through which the light beam passes during a measurement may change the spectra in an uncontrolled manner. Such change complicates the mathematical subtraction of the background, making it difficult and/or unreliable. In a preferred embodiment dry nitrogen gas is added to spaces where the infrared beam passes on the way to and from a sample.

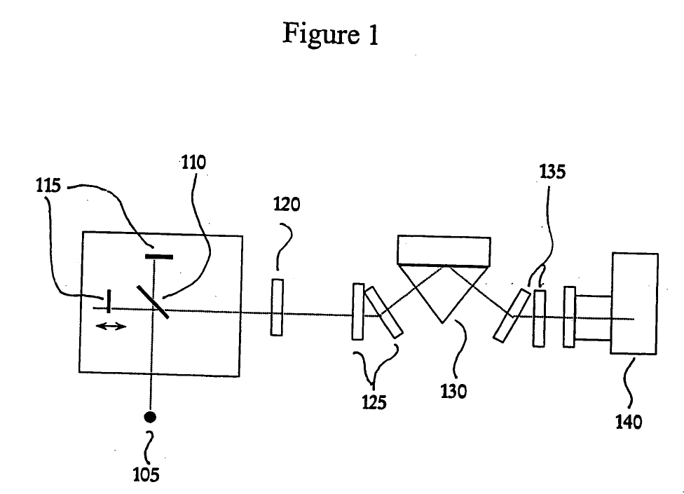

Representative Instrumentation. An example of a reflectance mode apparatus according to embodiments of the invention is provided in FIG. 1, which shows a light source, detector and some parts between the source and detector. Light from light source 105 passes through beam splitter 110 and is reflected by interferometer mirrors 115 into spectral filter 120. Light from spectral filter 120 is focused via focusing and beam steering optics 125 into the bottom of sample holder 130. The light then interacts with each sample in one or more passes and is then reflected out of sample holder 130 and is focused by optics 135 into infrared camera 140. An embodiment of this system as shown in FIG. 1 comprises five components: 1) source of infrared radiation, 2) a device to modulate the radiation, 3) a sample holder, 4) an infrared detector, and 5) a computer to collect, process, and present the spectral data.

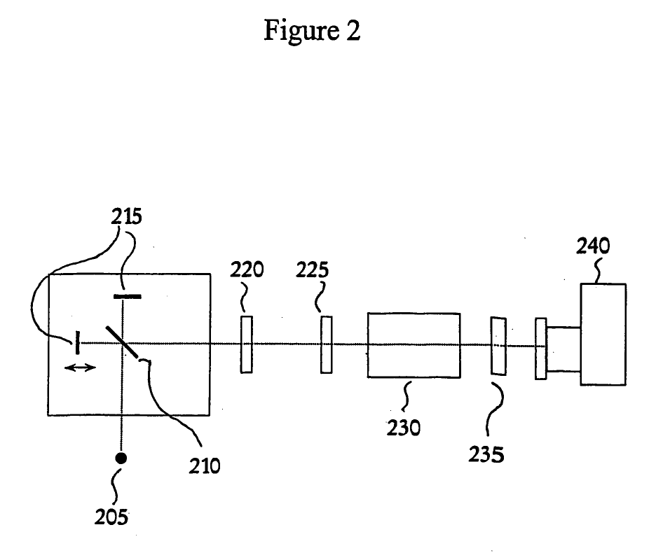

An example of a transmission mode apparatus in accordance with an embodiment of the invention is shown in FIG. 2. Here, radiation from source 205 passes through beam splitter 210 and is reflected by interferometer mirrors 215 into spectral filter 220. Light from spectral filter 220 is focused via focusing optics 225 into the bottom of sample holder 230, where each element of a sample array within holder 230 is illuminated simultaneously. Radiation passes through the samples and then is focused by optics 235 and enters infrared camera 240.

Transmission Mode Sample Holders. Transmission measurements are carried out by passing light from a source through a sample and to a detector and generally require different sample holders than that used for reflectance measurements. Solution based infrared transmission measurements generally require a short path length transmission cell or a flow-through cell. In both configurations the optical path length through the sample is restricted to short distances such as about 5-10 microns in length for aqueous solutions. A sample may be sandwiched between two infrared transparent windows separated by a thin gasket (Teflon) designed to confine the sample and fix the path length. A similar sample holder exists where the sample flows through a pipe with an infrared transparent sidewall to let light in and out. Neither configuration allows simultaneous acquisition of infrared absorption spectra from multiple samples. The problems of multiple transmission measurements in parallel can thus be stated as requiring: i) a separation of all samples in an infrared beam; ii) control of the required short path lengths; and iii) reduction of solvent evaporation.

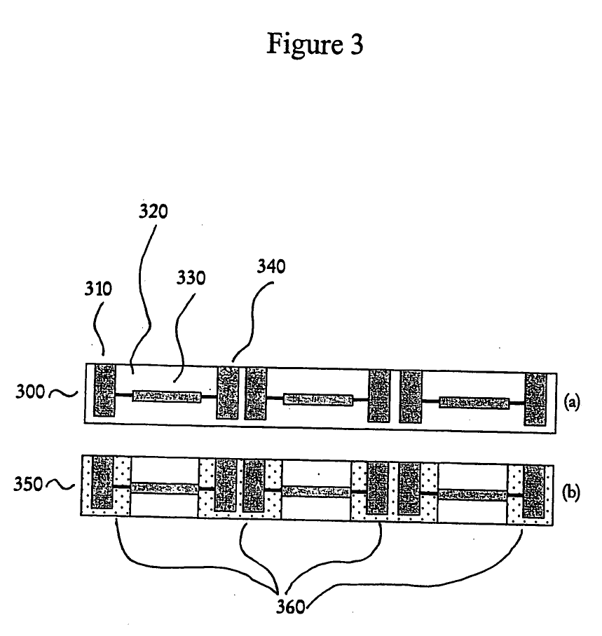

These problems were successfully addressed by the discovery of a parallel sample holder design as exemplified in FIG. 3. This sample holder has several features that alleviate these problems. First, the holder contains infrared transparent regions to let the beam pass through the sample. These infrared transparent sampling regions may be created by constructing the entire holder from an infrared transparent medium, or by integrating a series of infrared transparent windows into a non-transmitting matrix. Second, the sample holders contain specific sample injection ports, as seen in FIG. 3. Each sample location may have several sample injection ports to allow combination of reactants, solvents, etc. Finally, the sample injection ports are connected to the infrared sampling region by microchannels, which allow the sample to move from the port to the sampling region by capillary action. The capillary fed, short path-length sampling regions can be modified as suited to limit the beam path through the sample and isolation as needed to reduce solvent evaporation.

More specifically, FIG. 3a shows sample holder 300 having three sampling units constructed with infrared transparent material. As seen for the left hand most unit, sample injection/removal port 310 is used to add or remove a sample or a sample stream that flows through capillary micro channel 320 into sampling region 330 and then out sample injection/removal port 340. Sample holder 350 shown in FIG. 3b further includes non-transparent matrix regions 360.

The infrared transparent regions of these sample holders can be made of one or more infrared transparent materials such as an alkali halide salt (KBr or NaCl), CaF₂, BaF₂, ZnSe, Ge, Si, thin polyethylene, or specialized infrared materials such as AMTIR and KRS-5. The use of materials such as Si and Ge allow the entire sample array to be microfabricated using lithography and standard semiconductor processing techniques. The non-transmitting matrix can be made of a low cost material such as a plastic, glass, wax, polymers, elastomers, and so on.

Internal Reflectance Mode Sample Holders and Components. Further embodiments were discovered that provide superior attenuated total internal reflectance for multiple samples through use of new prismatic structures that direct light for optimum effect, as detailed below. To place these several discoveries in context, a brief summary of how attenuated total internal reflectance spectroscopy works is provided along with a description of how certain discoveries relevant to this procedure are used. Based on this information, a skilled artisan may further optimize the presented embodiments for particular sample arrangements.

Many embodiments of the invention use attenuated total internal reflectance spectroscopy for samples that are very strong infrared absorbers, such as water. In these embodiments a beam of energy entering an optical cell undergoes total internal reflection at the interface between the sample and the optical cell when the angle at which the incident light impacts the sample/sample holder interface is greater than the critical angle. The critical angle is material dependent and based on Snell's Law. The angle is defined by the indices of refraction for the sample and the optical cell. This angle is particularly important to the dimensions and placement of prismatic structures according to embodiments of the invention. This is because a surface skimming (evanescent) wave is created when light impinges at the fluid sample surface at or above the critical angle. This surface-skimming wave reacts with the sample in close proximity to the interface between the sample and the optical cell, and then exits the cell.

Prismatic structures as discovered and described below are dimensioned for and positioned to control probing light to enter a sample/sample container boundary so as to enter the boundary at an angle equal to or (more preferably) greater than the critical angle. In an embodiment a prismatic structure controls a probing light spectrum beam to enter a sample/container boundary within 2 degrees of a critical angle determined for light of wavelength in the middle of the beam spectrum. In another embodiment the light beam is controlled to enter at 0-2, 0-5, 0-15, 0-30, 0-45, 5-10, 5-15, 5-30, 15-45 or even 0-45 degrees greater than the critical angle. In another embodiment a prismatic transparent structure is dimensioned and positioned to direct a probing light spectrum beam to enter a solid medium at an angle that is more perpendicular to the solid medium, in order to minimize reflective losses.

The penetration of a probing light spectrum beam into a sample is short, typically on the order of the wavelength of the incident light (Equation 1). This eliminates the need to control and measure the path length or volume of the sample during the measurement. This feature allows more convenient and inexpensive use of multiple sample arrays that may be placed in optic communication with the probing beam and a detector with less need for precision. Heavily absorbing samples with thicknesses greater than 1 mm can be measured with this configuration.

$$Penetration \frac{λ / {n}_{1}}{{D}_{p} = 2 {π [ {sin}^{2} θ - {( {n}_{1} / {n}_{2} )}^{2} ]}^{0.5}}$$

Such optical cells may be fabricated into individual crystals and preferably, for infrared measurements, are made of infrared transparent materials such as, for example silicon, germanium, zinc selenide, AMTIR, and KRS-5.

The use of attenuated total internal reflection has been exploited by others and a variety of components useful for building instruments are available. However, generally, such instruments have been strictly limited to the use of single samples. For high throughput analysis of many biological samples, prismatic structures and other features as detailed herein are combined, as discussed next.

Prismatic Designs for High Sample Throughput. The inventors discovered several prismatic designs, devices and methods of their use that greatly improve the capability of total internal reflectance assay of large numbers of samples in parallel. Generally, the prismatic properties of carefully dimensioned optic devices are chosen to control the probing light (light that contacts the sample interface for reflectance measurements) to enter a sample interface at a suitable angle. Embodiments of the invention provide devices wherein probing light simultaneously enters multiple sample/well interfaces at different locations. Moreover, the devices maintain approximately the same (i.e. within 10 degrees, preferably within 5 degrees, more preferably within 2 degrees and preferably within 1 degree of a standard deviation) incident light angle into each sample. Preferably, the incident light angle is greater than the critical angle throughout the entire array.

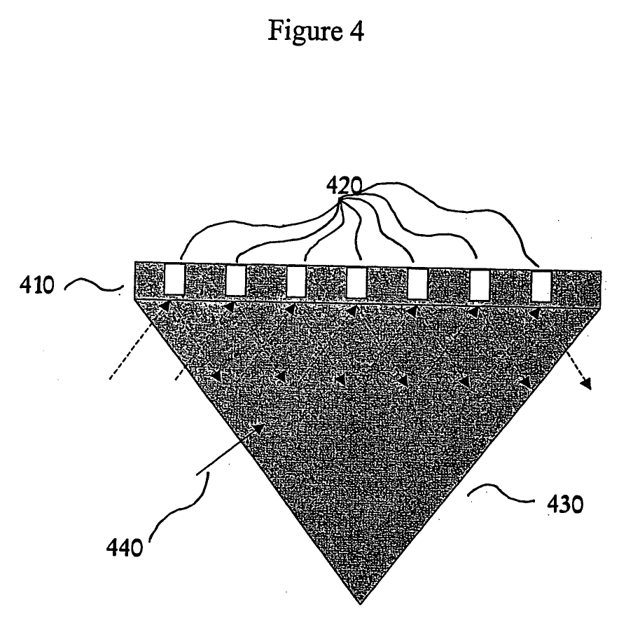

Three types of advantageous sample holders for attenuated total internal reflectance measurements were discovered. A first type maintains substantially equivalent optic treatment of multiple samples through optical contact of a large optic device that acts as a large common prismatic device for all samples. The large optic device directs light into and out of each sample simultaneously. In a preferred embodiment the outer surface of the large optic device is dimensioned so as to direct light simultaneously into sample wells at an incidence angle that equals or exceeds the critical angle, as seen in FIG. 4.

FIG. 4 shows the use of a fabricated array of attenuated total reflectance sample holders 410. Array 410 consists of sample wells 420 capable of holding fluid, particularly aqueous liquid, or solid samples. Optical interface prismatic device 430 contacts the bottom of array 410 and is capable of directing infrared radiation beam 440 from outside to the interface with the sample under appropriate conditions for internal reflection. In the arrangement shown in FIG. 4, infrared radiation beam 440 is incident on the surface of transparent prismatic device 430 and enters holder 410. The infrared radiation interacts with sample wells 420 and the other sample wells and then reflects away from sample holder 410 as depicted in the broken lines.

In a desirable embodiment large prismatic device 430 is a fixed part of the optics of an instrument and array 410 is separable, allowing sample processing and manipulation away from the machine. In an embodiment array 410 comprises infrared transparent material and is re-used. In another embodiment that uses visible light, array 410 comprises visible light transparent material such as an inexpensive plastic and is disposable. A preferred sample array device is a microtiter plate, particularly having 96 wells or a multiple of 96 wells such as 384, 1536 or 3072 wells. A preferred size of an array is for a length of approximately 108 millimeters and a width of approximately 75 millimeters. In an embodiment the sample array is processed by robotics used for regular microtiter plates having at least 96 wells.

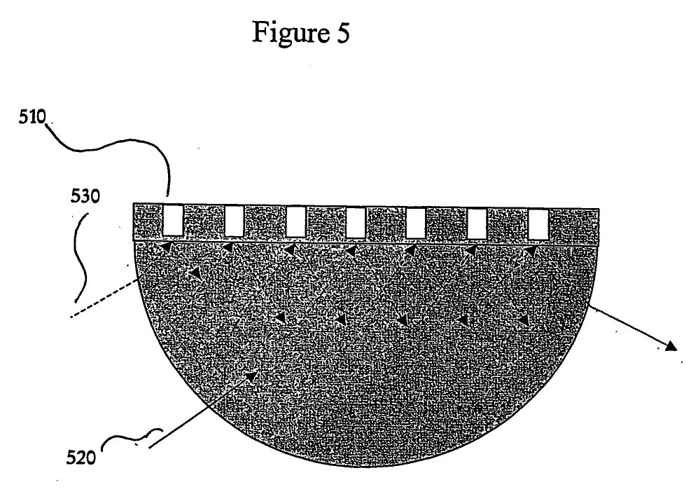

A variety of shapes and dimensions may be used according to the discovered structures and features described here. For example, FIG. 5 shows a sample array 510 that is in contact with a large prismatic hemisphere 520 having a curved surface that bends probing light beams (see light beam 530) to enter the array such that the light beam meets or exceeds the critical angle at the surface between the sample and the transparent material of 510 in contact with the sample.

As exemplified in FIGS. 4 and 5, a transparent prismatic material shown as 420 and 520 in these figures, respectively may be the same material as that use for the sample holder. A prism or hemisphere shape as shown in these figures is particularly advantageous but other shapes may be used. In the embodiments of FIGS. 4 and 5 the transparent prismatic material contacts the flat bottom of the sample holder and directs light beams that typically are parallel and enter different sample surfaces in a reproducible manner.

The reflection/transmission efficiency is strongly dependent on the angle of incidence between the light and the optical medium. A normal incidence results in the maximum transmission and minimum reflection. Therefore adding an optical structure such as that shown in FIGS. 4 and 5 allows light to enter the optically dense material at near normal incidence for reduced reflective losses and increased light throughput to the sample. The transmission efficiency can be further improved by adding a broadband anti-reflection coating to the optical structure.

The prismatic or hemispheric optic (structures 430 and 520 in the figures) can remain in the instrument allowing the sample holder to be removed and replaced. The interface between the flat bottom of the sample holder and the prismatic or hemispheric optic is not perfect, meaning there may be a slight air gap due to non-uniformity. This gap would represent a location for further reflective losses. To overcome this, an index matching fluid is used to fill the gap. Most advantageously the refractive index of the fluid has a numeric value that is between the refractive indexes of the two material surfaces and more preferably is midway (within 20% of the mean value), between the two solid phase refractive indexes.

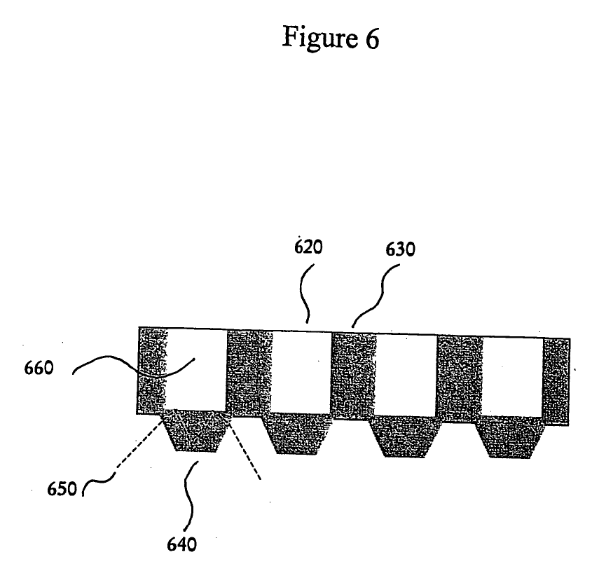

A second type of geometrical arrangement uses separate prismatic portions adjacent to each sample surface to direct light beams to interact with each sample in multiple locations before reflecting away from the sample holder. A prismatic sample array according to this second type is shown schematically in FIG. 6. Array 610 has wells 620 and walls 630. Walls 630 advantageously are opaque. Lower optical portion 640 of the left most sample allows light beam 650 to enter and undergo two reflections off lower sample surface 660 before exiting.

Typically the lower surface of a sample well establishes an attenuated total internal reflection with the sample and an adjacent optical portion (which may be continuous with the surface in contact with the sample or may be separate) is prismatic. In this case the lower surface ideally is a prism with a high aspect ratio, i.e. long length and short thickness. Advantageously the aspect ratio exceeds 1.5, 2, 3, 4, 5, 7 8, or even 10. The prism allows the light to enter at near normal incidence for the reasons discussed above. Light becomes trapped between the upper and lower faces of the prism, similar to a fiber optic.

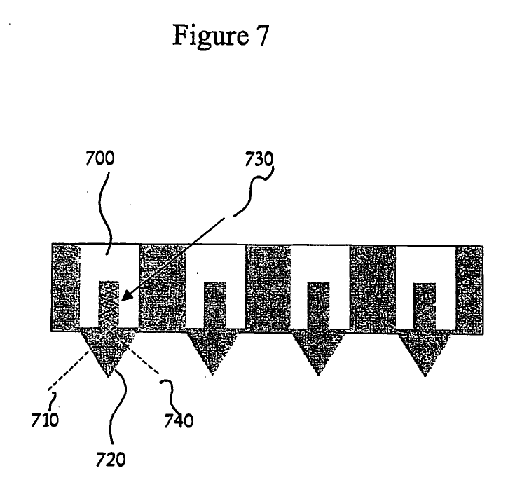

In the third type of geometrical arrangement a multiple internal reflection element protrudes into the sample well from an optical surface, and a prismatic optical surface is created at the optical surface. A representative array of multiple internal reflection elements is shown in FIG. 7. As seen in the left hand sample well 700 of FIG. 7, light beam 710 enters prismatic optical surface 720 on the lower optical surface and propagates up into multiple internal reflection element 730 where the radiation interacts with the sample at multiple locations before exiting as beam 740 from the prismatic surface on the lower optical surface.

Combinations of the three types of geometrical arrangements may be made and various dimensions will be appreciated by a skilled artisan.

Multiple Sample Holder Geometries with Internal Reflection. Many of the embodiments thus far described employ unitized optics that manage multiple light interactions within a common optic with multiple samples exposed to the optic. Further designs were discovered that exploit new sampling modalities based on internal reflection within individual spheres and within individual light conducting fibers. In an embodiment each sphere and/or each fiber represents an individual sample. Large assemblages of spheres may be used simultaneously in suspension and assemblies of fibers may be used for large scale sampling.

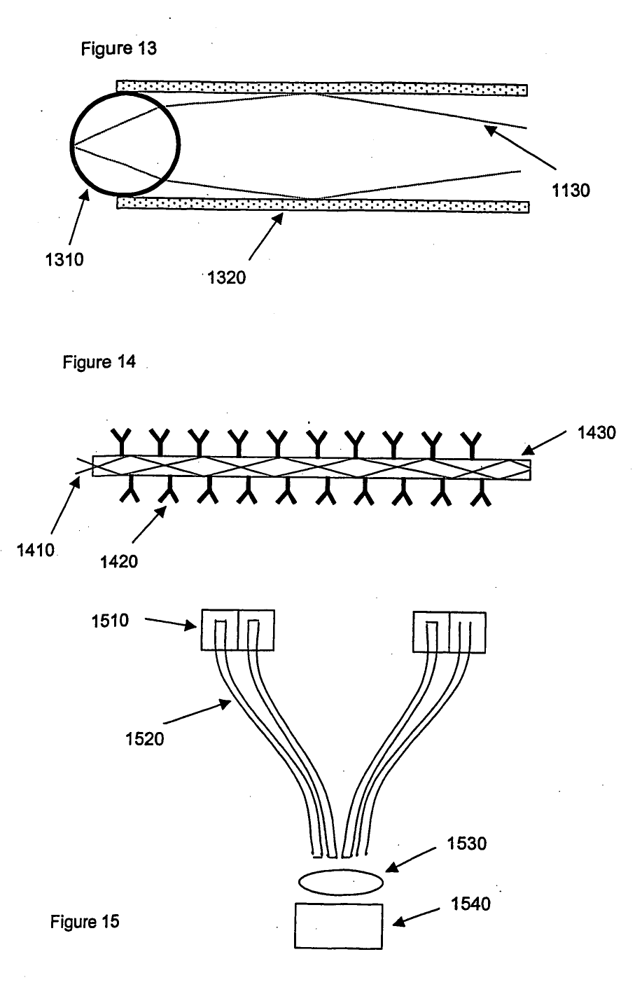

Internal reflection may be carried out in a sphere as exemplified in FIG. 13 for a proximity assay. This figure shows a sphere made of an infrared transparent medium with an index of refraction greater than a surrounding solution. This combination combines attributes of conventional proximity assays (such as Scintillation proximity assays SPA) with spectral identification in an internal reflection geometry. FIG. 13 shows a schematic representation of a contemplated proximity assay. In this embodiment, sphere 1310 made of an infrared transparent medium of high refractive index is covered with a biological capture layer (not shown), such as an antibody, that specifically can bind to a protein or other analyte in solution. Multiple spheres are mixed in solution and allowed to capture the desired analyte. Following analyte capture a sphere is immobilized, for example at or in the end of an infrared transparent optical fiber 1320 or in a tube or structure of transmitting infrared radiation 1330 that allows the radiation to propagate from the source down the pipe, into the sphere, where it is reflected back out and up the light pipe for detection. The sphere enables internal reflection sampling as described earlier.

The spherical capture assay provides, in many cases a more natural environment for binding reactions and more favorable binding kinetics due to improved diffusion of particles in suspension compared to that of immobilized binding partners on a large solid surface. Furthermore, the bead enables internal reflection sampling as discussed before. Still further, the small size coupled to a capture technique allows use of multiple spheres, which may be coated with similar or different biological capture surfaces. This controlled diversity enables identification of specific analytes and/or interactions with specific spheres.

Sphere size also may be controlled to differentiate among spheres by the capture technique used to immobilize spheres from solution. Immobilized spheres may be assayed in their original binding solution, for most natural conditions in an embodiment. In another embodiment the spheres are washed and then captured. In yet another embodiment the spheres are immobilized on a filter that is made up of an array of fibers or tubes. The size of the sphere (sphere 1310 in FIG. 13) relative to the matching light pipe such as a tube (tube 1320 shown in FIG. 13), fiber or other structure enables specific sphere types to be captured by corresponding light pipes. This is particularly advantageous for use with different sized spheres that contain respective different coatings. The other end of these pipes can be bundled up to a common collection point where they can be imaged simultaneously with the hyperspectral imager. In an embodiment fibers are used and each end is cupped to accommodate a particular sized sphere for internal reflection measurements.

In an embodiment cross-sectional dimensions of a light pipe allows the light pipe to perform like a waveguide. The dimensions of the waveguide control the range of infrared wavelengths that can propagate down the light pipe. In other words infrared radiation with a wavelength greater than the waveguide's cutoff wavelength cannot propagate in the light pipe. Accordingly, different light pipes can be used to sample different regions of the infrared spectrum, as defined by the dimensions of the light pipe. Since the size of the sphere is matched to a light pipe for capture purposes, different size spheres can participate in sampling at different respective regions of the infrared spectrum. In another embodiment light pipes are used that comprise materials of different optical band pass characteristics. A skilled artisan readily can appreciate how materials and dimensions of light pipes and/or tube and/or other structures may be altered to achieve particular combinations of optimum wavelength transmission.

Internal reflection may be carried out with a fiber. The fiber may be coated with one or more molecules that participate in binding reactions and can be used similarly as described for spheres. Infrared transparent fibers can be coated through a large variety of techniques. The dimensions of each fiber may be controlled to facilitate internal reflection. Furthermore, fibers may be bundled to simultaneously measure multiple samples using hyperspectral imaging at the end of the fiber bundle. The same size considerations, material considerations and the waveguide nature available for fibers apply in this geometry.

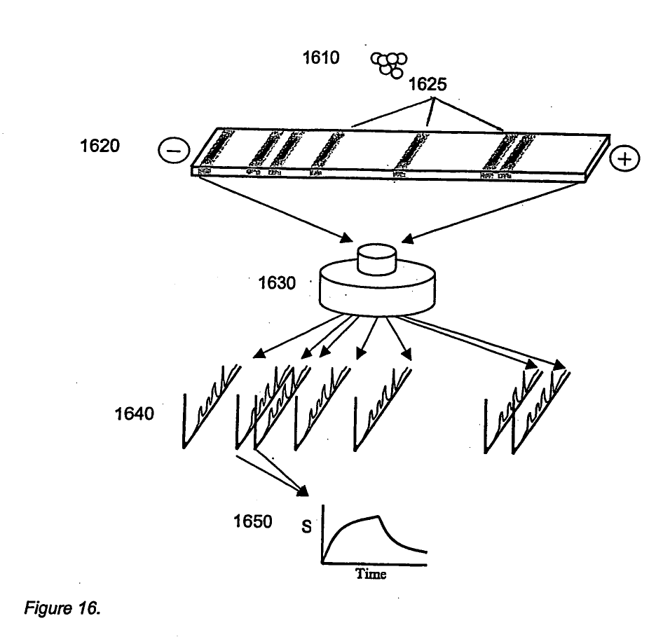

FIG. 14 illustrates a coated fiber embodiment wherein fiber 1430 is coated with antibody 1420. Light beam 1410 undergoes multiple internal reflections, which are resolved by hyperspectral imaging. Fibers can be bundled into a multiple assay device. Furthermore, more than one multiple fiber assay device may be used sequentially or at the same time with optics and hyperspectral imaging. FIG. 15 shows a representative example wherein multiple fiber optic sampling devices 1520 collect spectral information from multiple samples 1510 simultaneously. Infrared optics 1530 passes light into and out of the fibers for simultaneous measurement by hyperspectral detection system 1540.

Further Advantageous Features

Spectral Filtering of Source Radiation. A majority of contemplated applications require accumulating spectral information in the wavelength range between 5-16.5 microns (1 micron=10⁻⁶ meters), or between 2000-600 cm⁻¹. Infrared sources emit radiation over a large wavelength range from the visible to the far infrared and embodiments of the invention use the various wavelengths. Infrared wavelengths outside a desired spectral window may adversely affect the measurement through sample heating. Uncontrolled heating in turn causes background (baseline signal) drift and decreases signal to noise ratio of measurements. Therefore, a spectral filter preferably is included to limit the infrared radiation from a source to a bandwidth of interest, and blocks other radiation generated from the source but which is not necessary for a measurement.

Such blocking is particularly valuable when light intensity is increased for small area samples (i.e. high power density applications). An infrared filter can be fabricated by deposition of a thin film(s) of specialized material(s) (metals and semiconductors) onto a infrared transparent substrate. The thin film layer(s) alters the transmission and reflection coefficients of the optical interface such as to limit the bandwidth of radiation allowed to pass through the filter. A general discussion can be found in many optical texts, at http://www.ocli.com/pdf_files/products/gen_info_infrared_filters.pdf or in O. S. Heavens Optical Properties of Thin Solid Films 1991, Dover Press, New York.

Polarization Filters for Enhanced Performance. Advantageous embodiments of the invention exploit the polarization properties of light to obtain additional information from the total internal reflection spectrum using a polarizing filter. In an embodiment a linearly polarized filter is placed in the beam path in front of the detector and thereby limits the infrared radiation entering the detector to a certain preferred polarization. Typically, the polarization is adjusted to allow either radiation polarized in the plane of reflection or perpendicular to the plane of reflection in order to enhance the signal of oriented thin film samples relative to the background. A photoelastic modulator optionally may be used to rapidly modify the polarization between left and right circularly polarized states, generally between 10 Hz and 10 MHz, preferably between 100 Hz and 1 MHz and more preferably between 1 KHz and 100 KHz frequencies. The detector measures the differential absorption of left vs. right circularly polarized infrared radiation. This differential polarization provides for the detection of stereochemical information, such as chirality. In an embodiment differential polarization allows the differentiation between enantiomers.

Modulation. Modulation, combined with Fourier transform analysis is particularly powerful for improving signal and analysis time. Light from the source preferably is modulated with an interferometer. A preferred interferometer is a Michelson interferometer. Numerous other interferometer designs exist and are suitable. In principle any interferometer that creates an optical path difference will work in one or more embodiments.

Camera Speed and Spectral Resolution. Many laboratory based mid-infrared imaging spectrometers utilize a Michelson interferometer to modulate infrared radiation before the radiation interacts with a sample. The Michelson interferometer often is used in commercial FT-IR spectrometers as the “light source” in their systems. The Michelson interferometer uses a moving mirror system to generate an optical path difference between two components of a split light source. The spectral resolution of a two-beam interferometer is based on the overall optical path difference in the interferometer and number of optical path differences at which the detector is read (number of mirror positions measured). The data from each of the optical path differences is converted to an absorption spectrum with the aide of a mathematical (e.g. Fourier) transform algorithm and a computer.

Two beam systems are capable of very wide bandwidths (25,000-13 cm⁻¹) and very high-resolution (˜0.005 cm⁻¹) operation, and are particularly described as they are useful in embodiments of the invention. The need to move one or both mirrors complicates time sensitive analysis when the kinetics of the event being measured is on the same time scale as the mirror speed. In other words, the data are averaged over the time needed to sweep one length of the mirror path; speed and resolution are inversely related. Certain two-beam interferometers utilize a step-scan configuration, where the interferometer steps to a fixed optical path difference and scans a small amount (small mirror movement) around that path length.

The influence on imaging systems is even more profound due to the increased time needed to get the data from the array. The array speed generally scales with the size, the smaller arrays being faster, and single pixel detectors (found in FT-IR spectrometers) generally operate at MHz frequencies. A typical 64×64 pixel Hg—Cd—Te array has a maximum frame rate of 420 Hz, with specialized arrays allowing operation at ˜1 kHz. Since an image must be taken for each optical path difference (mirror position), and the spectral resolution is dependent on the number of different mirror positions measured, higher resolution translates into longer times in the imaging sense as well.

Complicating the speed issue further, many chemical and biological reactions require numerous spectra that must be averaged for noise reduction prior to data processing. A typical protein experiment, for example, may require the combination of 100 or more spectra data for mathematical processing via one or more algorithms such as smoothing, derivatizing, curve-fitting, etc.). Embodiments of the invention provide rapid multiple spectra from each sample in an array which increases system performance and provides good sample throughput speeds.

Correction with an internal standard. One of the largest contributors to noise when taking infrared measurements in aqueous solutions is drift in the background (baseline). This problem may be addressed by generating a background (baseline) measurement and then using that measurement to reference subsequent spectra. In many cases the stored baseline spectrum is subtracted from subsequent spectra. Typically the baseline will change due to changes in temperature or changes in the atmospheric conditions, such as changes to humidity, carbon dioxide content, etc. These changes manifest themselves as an incomplete subtraction or overcompensation of background effects. The drift problem is acute for measurements of dilute concentrations of molecules, where the baseline noise may overcome the desired signal from molecules in solution.

One method to alleviate the problem is to take a new baseline before every experiment or every time the background has changed. In long reactions the background may change often, requiring many new baseline measurements. Two methods are particularly desirable to rectify this increased noise problem.

One method of correction is to use photolabile groups to trigger chemical reactions and time the background measurements according to the triggering time. For example, a new baseline can be determined before every triggering event. Adding up the spectra acquired after every triggering event produces a spectrum more free of baseline changes.

Another method is to place one or more reference samples in one or more sample wells within the sample array, and use spectra obtained from the well(s) as a baseline for other samples in the array. For example, one well in a 96 well plate is filled with solution similar to all of the other wells, but containing no test molecule(s). Spectra collected from this well are used as baseline (reference) spectra. Spectra collected from the other 95 samples in the plate are referenced to the baseline spectra from the reference well. This method utilizes the power of parallel data acquisition to utilize one or more wells within the sample array as a reference for background changes. This correction procedure is particularly valuable for acquisition of multiple spectra over long reaction times such as minutes or even hours.

Binning for increased speed or sensitivity. The slow speed of the infrared focal plane arrays is the primary limiting factor in data acquisition speed for this type of instrument. The parallel nature of spectral imaging slashes spectral averaging speed and compensates for the slower speed of the focal plane array. Generally, radiation from each sample in the field of view of the infrared detector strikes more than one pixel on the detector. Combining the intensity data from all such pixels for a given sample allows averaging of measurements from multiple single detectors, in a similar manner as described above for spectral averaging. For example, a typical data set from averaging 100 spectra can be obtained in 6 scans by binning the 16 pixels.

Binning is particularly effective for arrays of samples where the size of the sample array is smaller than the size of the focal plane array. A typical 384 sample array (16×24 samples) using a 64×64 element focal plane array would place approximately 5×2=10 pixels on each sample spot. Binning the 10 pixels reduces the required scans ten fold. A conventional FT-IR spectrometer may require 76 seconds to acquire 100 spectra at 4 cm⁻¹ resolution (mirror velocity at 1.58 cm/s). Replacing the detector with a focal plane array and binning the same 10 pixels would reduce this acquisition time to 7.6 seconds.

Generally, true imaging systems that work with true images are not able to exploit binning in this manner. Unlike the parallel array systems described herein, those systems need every pixel in an array to increase spatial resolution. Thus, the binning method described here runs counter to the needs in that related art. That is, improved acquisition speed for spectral averaging is specific to parallel data acquisition, and cannot be used where high spatial resolution is required. Preferred embodiments incorporate binning when gathering data and are at least ten fold faster than a non-binning machine. Where the sample array has more elements than the focal plane array, binning can still be employed if tiling is used, that is, through stitching together of multiple images.

Detectors. A parallel infrared spectrometer for these purposes should have a detector sensitive to mid-infrared radiation in the 5 to 17 micron wavelength range. These detectors include such materials as Hg—Cd—Te, DTGS, thermopiles, quantum well infrared photodetectors (QWIP's), as well as many types of cooled and uncooled bolometers. In an imaging or parallel spectrometer, these detectors are found in either linear (1×128, 1×256, etc.) or rectangular arrays (64×64, 128×128, 4×256, etc.). The detector and read-out electronics form the components of an infrared camera. The camera converts the incoming radiation into a spectral image using mathematical transform algorithms on a standard personal computer.

Sample Holders and Solvents for Infrared. A majority of chemical and biological reactions take place in aqueous or organic solvents that absorb mid-infrared radiation well. For example, strong absorption in the mid-infrared spectral region generally limits the optical path-length to 5-10 microns in aqueous solutions. Conventional one-at-a-time spectrometers typically use three approaches to obtain spectra in these environments. They include, short path length or flow-through cells, total internal reflectance, and solvent evaporation. Each approach is constrained by the need for infrared transparent sample holder(s), or at least regions in the holder that are transparent. Many embodiments described herein address this problem by (in comparison with earlier art) shrinking the sample size and assaying large numbers of samples simultaneously.

Infrared Light Sources and Focusing. Embodiments of the invention provide diagnostic signals obtained by interaction of light with chemical bonding electrons found in molecules of interest. The diagnostic signals form from electric impulses that correspond to detected light signals. A good signal to noise (random electrical background signals) ratio thus is important to obtain rapid measurements because as the measurement time decreases the amount of light processed (and the electrical signal obtained from the light) becomes smaller. Infrared light is used in many embodiments wherein desired spectral processes involve fundamental vibrational resonances of molecules in the mid-infrared region of the light spectrum, which generally is defined as 4000-400 cm⁻¹ (2.5-25 microns). A majority of biological compounds are limited to 1800-600 cm⁻¹ (5.5-16.7 microns).

To generate probing light in the infrared region, a blackbody emission source typically is used such as a “glowbar” (a hot material such as SiC), a sample or scene's intrinsic heat emission, or from solar infrared radiation. Preferred sources include a single glowbar (silicon carbide rod), Nernst glower (cylinder of rare-earth oxides) or an incandescent wire. A source typically may have power outputs of ˜50-100 W and a beam diameter of ˜4 cm, or a beam power density of ˜4 W/cm². This power density can be increased with focusing optics for smaller samples, and reduced when an aperture is placed between the source and the sample. This power density is acceptable for traditional infrared experiments that involve a single sample in the beam path, or small area samples where the beam can be focused to a specific spot. In larger area sampling environments that exist when hundreds of small samples are to be measured simultaneously, broadening the beam to increase the effective area decreases the power density at each location in the sample. Therefore in order maintain an advantageous power density for an increased area of larger samples the infrared source power desirably is increased.

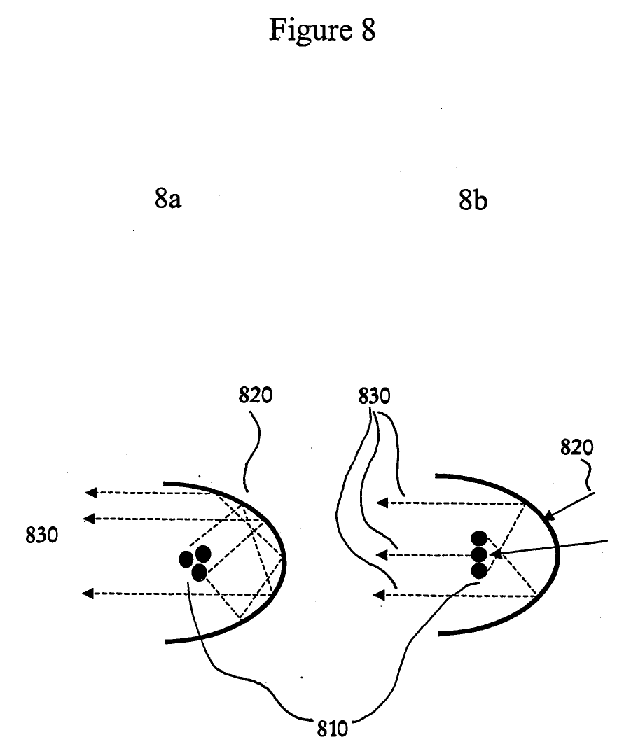

In one embodiment spectra are collected simultaneously from a “standard” micro titer plate format commonly used in the biological and chemical industry. These plates may hold an array of 96, 384, 768, 1536, 3072, 4608, 6144 or other number of sample wells. The relevant area of a titer-plate format sample holder is approximately 80 cm² and a source intensity of ˜350 W is necessary to produce the same 4 W/cm² at each sample location. This power optionally is increased to accommodate any apertures or optical losses in the beam path. A larger glowbar or Nernst source advantageously may be used for increased source intensity whereby a greater surface area on the source causes more emission. Alternatively multiple lower-intensity sources may be used in tandem. In the latter embodiment parabolic mirrors may be used to collect the light from several light sources and collimate it in the direction of the sample. Several embodiments of the latter are shown in FIG. 8. Three lower intensity sources 810 shown in FIGS. 8a and 8b may be positioned in a cluster or side by side, respectively, while allowing parabolic reflector 820 to direct the emitted light in a parallel fashion as shown by rays 830 in these Figures.

Biochemical and Cellular Focusing for Enhanced System Performance. The attenuated total reflection methods described herein rely on the proximity of a surface skimming wave (evanescent field) from a probing light to a sample. Biochemical focusing and cellular focusing techniques were discovered that exploit the functional separation of the sample volume into a probed portion (available to the surface wave) and a non-probed portion (essentially too far away to measurably affect the wave). In both focusing techniques greater signal to noise is achieved for improved measurements by physically concentrating the target to a sub-portion of the sample volume where the surface skimming wave is.

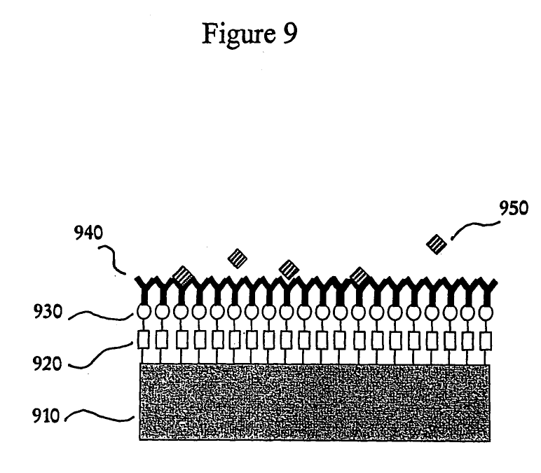

In biochemical focusing, molecule(s) are drawn out of solution and concentrate into a smaller volume that is exposed to a surface wave. The concentration step may be carried out by for example, a biochemical interaction layer or through an active process such as, for example, isoelectric focusing as described below. A representative embodiment of the former is illustrated in FIG. 9. FIG. 9 shows optical surface 910 upon which surface coupling ligands 920 are immobilized. Surface coupling ligands 920 are in turn attached to spacer ligands 930, and thence to capture ligands 940. Analyte 950, which typically is present throughout the liquid media binds to capture ligand 940. The binding of analyte 950 to the capture layer effectively brings more of the analyte within reach of the surface wave, and thus increases the effect of the analyte on the signal.

Each element in the sample array shown in FIG. 9 can have a similar or a different interaction layer tailored to a desired biochemical interaction. Such surface binding layers have been used in many biosensor designs, and are very common in diagnostic applications. Binding layers typically involve a ligand to bind the layer to the surface, various spacer or coupling layers, and a capture ligand or protein, such as an antibody. Different ligands may be chosen for coupling to the surface, depending on the type of surface. Ligands for coupling to silicon generally will differ from ligands that couple to polyethylene for example. U.S. Pat. No. 5,851,840 issued to Sluka et al. and also U.S. Pat. No. 5,240,602 issued to Hammen for example describe ligands generally and also ways to bind molecules to silicon surfaces. The use of capture ligands is well known, as for example described by U.S. Pat. No. 6,264,825 issued to Blackburn, et al., U.S. Pat. No. 5,658,732 issued to Ebersole et al., U.S. Pat. No. 5,498,551 issued to de Jaeger et al. and many others.

In cellular focusing, molecular interactions that occur at the surface of or within a cell preferentially are probed using a probing light wavelength chosen to match or exceed the size of a cell, which is to lie within the surface wave region of the sample. In this embodiment, cells adhere to the sample surface where the surface skimming wave is made. Preferably the mean wavelength of the probing light that generates the surface skimming wave is at least 5 microns, more preferably at least 7.5 microns, and in some embodiments more than 10 microns and even more than 15 microns. Preferably a cell is chosen having a thickness, (when lying flat on the sample surface) that is no more than twice the mean wavelength of the probing light and more preferably no more than the mean wavelength of the probing light. Preferential detection of molecular structures within cells, and at the cell surfaces are possible because the surface wave is chosen to be long enough to extend this far from the solid transparent medium but not long enough to cover most of the culture medium.

In another embodiment two types of probing light are used to discriminate between molecular events that occur close to the sample surface and those that occur further away. One light has a longer wavelength than the other and generates a more penetrating surface wave. For example, probing visible light of 600 nanometer wavelength may be used to obtain data for molecules and cell membrane surfaces that are attached to the surface. At the same time or at different times, probing light of 6000 nanometers may be used to obtain information for molecules and cell membrane surfaces that may be typically ten times further away from the optic surface.

Molecular Information from Isoelectric Focusing. A very desirable biochemical focusing technique useful for embodiments of the invention is electrophoresis, and particularly isoelectric focusing. The materials and methods described herein may be used to monitor molecular details of molecules that are separated by isoelectric points from isoelectric focusing as well as from other biochemical separation techniques. Embodiments provide information that allow the identification of specially separated molecules and of their interaction with binding partners. These embodiments provide significant advantages over conventional electrophoretic separation technology by revealing another dimension of molecular complexity in real time. For example, complex protein mixtures in their native environment may be separated by electrophoresis, and more preferably by isoelectric focusing, and analyzed without additional labeling with other moieties.

Advantageously, an instrument for hyperspectral analysis of isoelectric focusing in real time comprises a power supply, at least two electrodes in contact with the contents of a fluid sample holder, a source of broadband infrared radiation, a modulator of the broadband infrared radiation, an infrared radiation detector for detecting infrared radiation that exits the fluid sample holding chamber, and a computer for analyzing data from the infrared radiation detector. In the preferred embodiment of isoelectric focusing, during use, an aqueous solution that contains one or more ampholytes is exposed to a voltage gradient established by the electrodes and creates a pH gradient. A sample can, for example, be mixed into a solution that enters the holding chamber, or added at a separate time through one or more orifices. Two or more samples may be added at the same or at different times to the holding chamber, as may be desired to investigate intermolecular interactions spectroscopically.

The sample holder may be, for example, a linear one dimensional capillary, as is known in the art, or may be a two dimensional capillary or container. In another embodiment useful for increased sensitivity the holder has a section, preferably in the middle, having a small volume exposed to spectroscopic probing and larger volumes away from the probed volume. In a desirable embodiment the holder may resemble an hour glass, with a small probed volume and large volumes on each side to allow molecular concentration by isoelectric focusing prior to probing. Advantageously the sampled volume is 0.1; 0.03; 0.01; 0.003; 0.001; 0.0003; 0.0001; 0.00005 or even smaller with respect to the total sample volume and preferably the isoelectric focusing concentrates at least 10%; 25%; 50%; 60% or even more than 90% of the total amount of a desired molecular species into the sampling volume. Accordingly, spectra can be obtained from molecule(s) that may be at least 5, 10, 25, 50, 100, 250, 1000, 2000 fold or more concentrated during the analysis procedure itself. In another embodiment the sample holder contains a section, such as a thread, membrane, screen, or other form of immobilizing substance within it, to immobilize a certain type of molecular species, and facilitate spectroscopic analysis in three dimensions.

The instrument can take advantage of focal plane array detectors to simultaneously detect material arranged in a plane, and also multiple samples. Multiple samples may be arranged within a common infrared transparent holder, as for example, described herein. By eliminating the need for a support such as a gel, this embodiment of the invention allows for automated fluidic manipulation both prior to spectroscopic analysis and after. For example, samples from a microtiter plate can be sampled and a microtiter plate itself having optical features as described herein can be used directly. After isoelectric concentration and analysis, the analyzed material can be selectively (i.e. in a more purified form) or non selectively (entire sample) removed for further analysis such as mass spectrometry. It will be appreciated that much data can be obtained by such embodiments, as a) molecular concentration; b) molecular interactions; and c) isoelectric point separation can be arranged simultaneously, as well as multiple sample analysis for high throughput.

In a desirable embodiment high-resolution isoelectric focusing is used to separate proteins in a mixture according to their isoelectric points. Hyperspectral infrared imaging with light direction optics allows simultaneous whole-column monitoring of the focused protein. Generally, proteins in solution are introduced into a narrow capillary made from an infrared transparent material and an electric field is applied. Electric field induced movement of the proteins into sharp bands at the protein's native isoelectric point is monitored with a hyperspectral camera. Light may be processed and used in transmission mode or by using the internal reflection sampling mode.

Without wishing to be bound by any one theory for this embodiment of the invention, hyperspectral imaging of molecules while under the influence of an applied electric field overcomes the requirement and disadvantages of gel (or other) supports and staining. That is, a support such as a gel used in isoelectric focusing, is necessary to prevent diffusion when the electric field is turned off and allows staining to visualize protein bands. Protein bands are stained and analyzed in the absence of an electric field, which typically is shut off prior to staining. Without the electric field the protein bands being to blur as the protein diffuses. The presence of the gel limits diffusion of the protein. By removing the need to stain the protein the gel is no longer needed. Sharper bands, freedom from gels and other labels, and detection in real time are possible according to the desirable embodiments.

Yet another advantage of embodiments is that the infrared vibrational spectrum provides rich information about the protein, such as the protein's interactions with other molecules. Bound and unbound proteins for example, have different isoelectric points and thus assume distinct bands during focusing. Label free, whole column detection enables monitoring of binding events in real time, which can provide accurate information about binding kinetics. In many instances, binding is temporary, and the continuous application of an electric field allows monitoring of a shift in amount of bound protein back to an unbound protein band. Accordingly, association and dissociation kinetics can be determined in situ. Still further, chemical reactions that involve combining one or more substrates with an enzyme to produce a new product may be monitored in real time. One or more substrates that are non-charged may be present in the column media, may be introduced by another chemical reaction, or may be physically added to the column space. An enzyme or other catalyst focused at one location in the column space can generate a short or long lived product that can be seen or indirectly inferred (such as by protein conformational shift) spectroscopically. In an embodiment the substrate itself is visible light and/or ultraviolet light and produces a conformational shift or release of substance in a molecule or large molecular complex that is focused within the column space.

As described herein embodiments allow the identification of molecules and molecular complexes by physical separation parameters coupled with functional parameters, for greater resolution studies. Detection of new molecular parameters are made possible by the combination of hyperspectral imaging, particularly in the infrared region, with real time native protein interactions. By way of example, a mutated protein having an altered amino acid but that does not affect charge generally will not be resolved by separation techniques that rely on size or charge. However, any such mutation that affects a conformational change in the protein may be resolved by this technique. This new area of structurally silent mutations is very important to the burgeoning field of proteomics and embodiments of the invention are contemplated for large scale analysis of proteomics where a large number of protein alterations are made. By assaying conformational changes directly with hyperspectral imaging, rapid real time analysis of multiple samples is potentiated.

The ability to detect conformational shifts and other phenomenon of protein in its native environment allows the combination of high resolution separation with rapid reaction assays not readily found in other diagnostic tests. Furthermore, hyperspectral imaging may be used to deconvolute the presence of multiple proteins, even within the same band or spot, using advanced spectral processing tools. In this manner, the spectral fingerprint forms a second dimension of data in a “2D” separation. A particular advantage is that a protein may be held at its isoelectric point while capturing the vibrational spectrum, thus ensuing that the spectrum is that of the protein in its native environment. Thus, an embodiment improves over the previous art pertaining to simple detection methods such as refractive index, ultraviolet absorption, fluorescence and chemiluminescence to detect bands by extending detection to rich spectral information. In an embodiment a one dimensional procedure such as isoelectric focusing becomes more like two dimensional gel electrophoresis by providing a second dimension of data (infrared spectral fingerprint) with which to separate overlapping proteins, such as those with similar isoelectric points.

FIG. 16 shows an embodiment wherein hyperspectral imaging reveals molecular details such as location, movement, and binding of solutes from sample 1610 introduced to isoelectric separation chamber 1620. Bands 1625 form in chamber 1620 by isoelectric focusing. Infrared optics and detector 1630 simultaneous image bands 1625 to generate signal patterns 1640. The signal patterns are used to determine spectral changes that occur in time as depicted by graph 1650. The ability to carry out hyperspectral measurements in real time allow new types of isoelectric focusing that do not rely on high density, viscous or gel like matrices. For example, a complex two dimensional pattern can be established, in a bull's eye configuration with annular rings around a center electrode for assay of multiple samples. The system may be combined with a counter current flow of solute, binding partner, or substrate that may be constantly replenished or expose a focused sample to a periodic or other varying concentration to determine the effect of other substances including enzyme substrates on conformational spectra. This embodiment is particularly useful for drug discovery in instances where a test compound is consumed during reaction with an enzymatic molecule or macro molecular complex.

Manufacture by Semiconductor Processing Techniques. In an embodiment of the invention small arrays for simultaneous assay of many samples are prepared from semiconductor substrates. Such total internal reflectance arrays can be fabricated with standard lithographic processing found in the semiconductor industry. For example, one might use anisotropic wet etching of silicon or germanium and a photoresist to create prismatic features on a silicon substrate.

Suitable manufacturing techniques are described by, for example, U.S. Pat. No. 4,891,120 issued to Sethi et al., and other more recent U.S. patents, U.S. Pat. Nos. 6,331,439; 6,306,272; 6,245,227; 6,210,986; 6,180,536; 6,176,962; 6,158,712; 6,093,330; 6,033,628; 5,980,704; 5,872,010; 5,858,804; 5,585,069; and 5,194,133. Laser ablation techniques also may be used to make these devices as described in U.S. Pat. No. 5,658,413 issued to Kaltenbach et al. on Aug. 19, 1997. A good general summary may be found in Silicon Micromachining (Cambridge Studies in Semiconductor Physics and Microelectronic Engineering, 7), M. Elwenspoek, H. V. Hansen, Cambridge University Press (Cambridge), 1999.

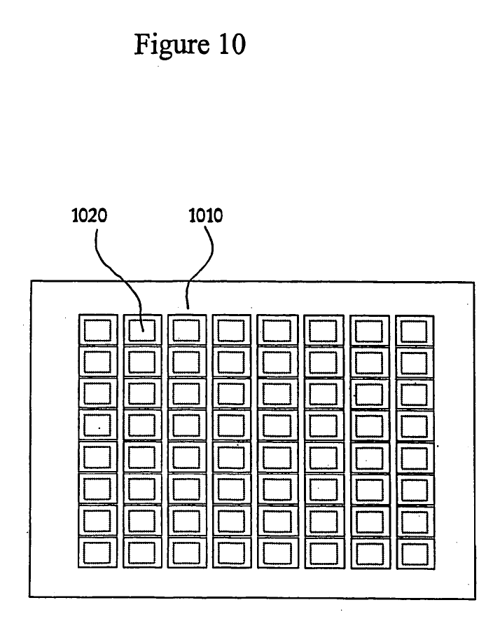

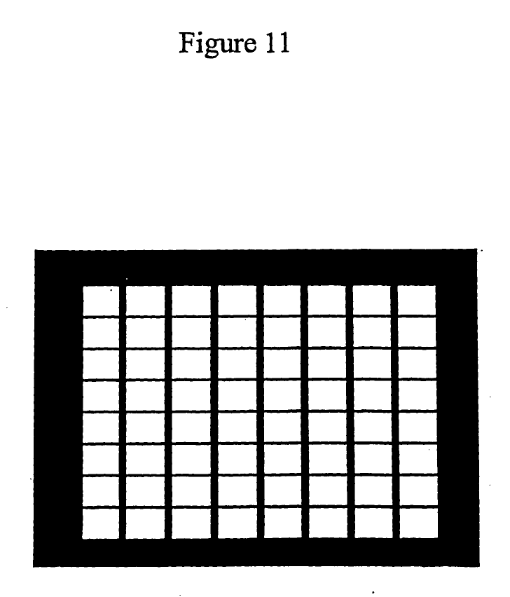

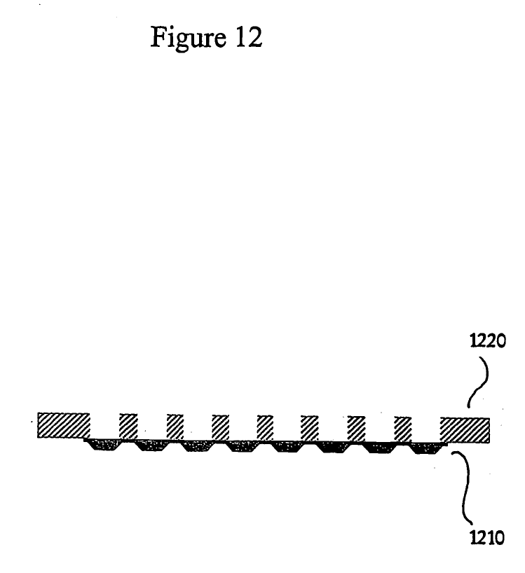

As an illustration of this embodiment, a 64 well sample array shown in FIG. 10 is made by the following procedure. Standard photolithography is used to transfer the pattern for the etch mask shown in FIG. 11 to an oriented silicon substrate. Then, vapor deposition is used to create the etch mask. The etch mask is a thin film of a nitride but other materials such as an oxide may be used. Nitrides are preferred for the long etch period used to produce the deep grooves. In this example, the silicon pattern as shown in FIG. 11 etches at an angle of 54.7 degrees, creating prismatic grooves and pits. The silicon is etched with KOH although other anisotropic etchants may be used as well. At this point the lower prismatic surfaces are formed. The sample wells next are created in plastic and then wafer bonded to the top surface of the etched silicon component. The completed sample holder shown in the top view of FIG. 10 has 64 well openings 1010 with narrower bottoms 1020. The side view of FIG. 12 shows a lower section of silicon 1210 and an upper section of plastic sample wells 1220. Many materials, such as glass, or metal may be used for the sample wells. Optionally, vertical structures, such as the multiple internal reflection elements, can be created with either DRI etching (reactive ion etching) or isotropic wet etching. In advantageous embodiments photolithography techniques are used to form larger arrays of at least 96 wells, 384 wells, 1024 wells, 5,000 wells, 10,000 wells, 25,000 wells, 50,000 wells, 100,000 wells, 250,000 wells, 500,000 wells and even more than 1,000,000 wells.

Internal elements are very useful for total internal reflection measurements and may be constructed by a variety of methods. One embodiment is a manufacturing process for making a large scale array of samples with total internal reflectance elements in them by binding one or more columns of polymer such as a plastic or protein to the inside surface(s) of each sample well. This embodiment is particularly useful for measurements using visible light. Many polymers do not absorb well between (for example) 400 and 800 nanometers and are useful for this embodiment. Total internal reflectance measurements may be carried out using a fluorescent probe and are particularly useful for this embodiment. The optical properties of the polymerized column may be corrected for and the system may be used in the near infrared, near ultraviolet, far infrared or far ultraviolet regions as well.

A polymer used to construct an internal reflectance element preferably is in the form of a rod between 0.5 micron and 100 microns wide, more preferably between 1 micron and 50 microns wide and even more preferably between 2 and 25 microns wide. The rod preferably has a length at least twice, three times, five times and even more than ten times the width (mean measurements). The rod is at least partially transparent to the light being used and can be a natural product, a synthesized product, or even polymerized before or during the analysis. Many plastics are known but natural materials such as proteins may be used. Preferably, thermostable polypeptide(s) are used. Available materials include natural proteins such as elastin-, collagen-, keratin-, and silk-type proteins, preferably, proteins derived from thermophilic bacteria such as Sulfolobus solfataricus and Thermus aquaticus (enzymes such as proteases, DNA polymerases, lipases, and metabolic enzymes are especially useful), and more preferably, synthetic protein polymers, particularly proteins designed with silk-like protein, SLP blocks (SLPF or FCB-SLPIII (fibronectin), SLPL (laminin), SLPC (cystine), SLP3, SLP4, and SELPs (elastin) as described in U.S. patent application Ser. Nos. 609,716 and 114,618, and peptides designed with SLP blocks and other materials as described in U.S. Pat. No. 5,723,588 issued to Donofrio et al. The polypeptides may be natural, chemically synthesized, or recombinant proteins, including modified forms such as mutants and fusion products, and also including modifications against thermally induced degradation or denaturation, for example, pegylation. The proteins may be polymerized on the inside of the sample wells, or may be attached to those surfaces by covalent or non-covalent binding techniques.

In a particularly desirable embodiment samples in an sample array are manufactured having internal prismatic structures that are optically and/or physically coupled to a semiconductor foundation and have a dimension that extends away from that foundation of at least 10 microns long. Preferably the length (extending away from the surface) is at least 1.5 time as long as the width, and more preferably at least 2 times, 3 times 5 times and even at least 10 times the width.

Super Broad Band System: Include Visible and Infrared Light. In another embodiment of the invention, attenuated total internal reflectance spectroscopy is carried out on samples with both infrared and visible light. A single broadband beam that encompasses infrared and visible light may be used that is modulated by an interferometer. Alternatively a infrared light may be used as described herein and a separate visible light source beam additionally may be trained upon the sample holder. In the latter case, a common prismatic structure may be used for both infrared and visible light beams. The two beams may be directed into the prismatic structure at different angles to accommodate differential bending of light due to wavelengths.

In an embodiment, a spinning mirror interferometer, such as that used for infrared measurements is modified for an increased mirror rotational speed as necessary for the shorter wavelength light. Advances in light modulation technology in the future will provide more convenient alternative methods for generating suitable modulation and are contemplated for embodiments of the invention.

Fluorescence, phosphorescence, time resolved fluorescence and/or chemiluminescence may be used in conjunction with infrared techniques as described here. Drug discovery methods advantageously may utilize such added information to reveal further molecular and metabolic information. The additional information is helpful particularly for biochemical and cellular studies where the effects of a test compound in a sample are very complex and multiple chemical interactions need to be examined. For example, a cell may be genetically engineered to express luciferin and luciferase and generate light from a biochemical pathway and used as a probe in multiple sample wells to test for new lead drug compounds. Effects from the test compounds may be detected as visible light signals. By monitoring both infrared reflectance and visible light signals simultaneously, chemical binding of test compounds to a cell surface can be monitored, and the timing and effect on the biochemical process monitored. In one such embodiment a prismatic device may be used underneath a sample array for infrared reflectance measurements and an imaging visible light detector may be placed above the sample array to monitor the location and intensity of light obtained from the array. The detected visible light signals and the reflectance spectroscopy signals are processed and compared to generate information pertaining to each sample.

Timed addition. In another embodiment a test substance or a chemical that controls a reaction may become available in the sample well from light activation. For example, a compound or set of compounds may be released by ultraviolet light acting upon a light sensitive labile chemical bond. Advantageously, a test compound is present, for example, on a wall of the sample container, and is released by a photoactivatible event. This embodiment is particularly useful for very large sample microassays of small size, wherein each individual sample well is very small (typically less than 10 microliters, less than 2, 1, 0.1 or even less than 0.01 microliters volume). The use of light activated chemistry in this embodiment alleviates the problem of having to administer test substances in very small volume at defined times. Light sensitive chemistries suitable for this embodiment are known. U.S. Pat. Nos. 5,798,491 and 5,714,328 issued to Magda et al. on Aug. 25, 1998 and Feb. 3, 1998 respectively, and a review by Keana and Cai in J. Org. Chem. 55: 3640-3647 (1990) provide representative examples. In practice a combinatorial library of molecules is generated and members of the library coupled via photo labile bonds to other molecule(s). The test molecules are liberated upon ultraviolet radiation.

An embodiment uses sample holders as described herein for a method of timed addition as follows. The surfaces of sample wells are divided into at least two surface coating types, a lower surface and one or more upper surfaces. The lower surface is within reach of a probing light used for total internal reflection. Preferably the lower surface in contact with the prismatic structure below extends upwards at least one wavelength distance of the probing light. The upper surface is too far away for significant optical interaction by a probing light evanescent wave. The upper surface(s) in an advantageous embodiment has attached to it a test substance or another activating substance that desirably is added to the sample solution at a defined time. That is, at the beginning of a test period, the test substance or other activator (compound or particle) is immobilized to an upper region of the wall and substantially out of reach of the probing light (which contacts the sample holder bottom). When a test is started, the immobilized substance(s) are released and their effects determined. The immobilized substances may be released by, for example, light catalyzed breakage of a link to the wall, sonication, change of air, electric discharge, or magnetic field. The released substances dissolve or become suspended in the sample fluid and can interact with components, such as cells and other molecules found there. The effects of the time released substances may be detected by total internal reflectance spectroscopy, using visible light, ultraviolet light or infrared light.

Definitions

To assist understanding of the embodiments and of the attached claims, the following definitions are provided.

The term “molecular interaction” means any interaction, including binding and catalytic interactions between at least two molecules. Binding interactions include for example binding between antibody binding site and antigen, binding between a protein and a ligand, such as between a membrane protein and an effector that binds the protein, and interactions determined indirectly by intracellular changes that occur upon addition of chemical substances that may act by binding to a cell membrane receptor, binding to effectors that bind to cell membrane receptors, thereby preventing effector binding to their receptors, and intracellular entry of a molecule that leads to some detectable change in another molecule or cellular process.

The term “wet samples” means samples that are in a fluid. The fluid may be an aqueous sample such as water, buffered saline, blood, interstitial fluid, sweat, urine and the like, but also may be non-aqueous such as xylene, dimethyl sulfoxide, dimethyl formamide, hexane, triglycerides, an alcohol and the like. Some binding reactions and some catalytic reactions have been studied using all organic (non aqueous) phase and such chemistries may be employed as well as aqueous chemistries. Gas phase reactions also may be included, where a binding molecule on the hydrated surface may bind to a volatile molecule. The concentration of and binding to an immobilized receptor or other binding substance on the surface of a material that experiences total internal reflection is included in the term “wet samples” as the surface of the well generally is hydrated.

The term “broadband infrared radiation” means multiple wavelengths suitable for determining a spectrum. Generally at least 3, more preferably at least 5 and even more preferably a large number of distinguishable wavelengths are included in the radiation. Preferably the bandwidth is at least 0.5 microns, more preferably at least 1 microns and even more preferably at least 2 or even at least 4 microns.

The term “optical interface” means having a defined solid structure that directs light such as probing light that enters one or more sample wells or positions, or modified light that leaves one or more sample wells or positions. An optical interface typically is an infrared transparent surface such as a prismatic portion of a larger surface or a separate optical structure that is positioned adjacent to or near another optical structure such as a sample holder or light source. The optical interface may for example effect the light passing through it to facilitate multiple internal reflectance in the optical interface or in another optical device. The optical interface effects light by virtue of one or more characteristics such as its refractive index, dimensions, and/or surface angle with respect to incident or exiting light.

The term “sample well” means a definable surface on or volume in an infrared radiation transparent material that holds a sample. A sample well may be a well of a 96 well microtiter plate wherein the bottom surface only is infrared radiation transparent. A sample well may be a three dimensional region corresponding to a position in an array that has been etched from a semiconductor chip surface. A sample well may be a surface having an immobilized substance. A flat surface without walls may nevertheless form an array of sample wells by virtue of chemical bonding that extends up from the surface immobilant. For example, a flat hydrophobic surface may be prepared by immobilizing a binding partner such as proteins, cells or other hydroscopic material in an array. Each binding partner is immobilized, for example as a hydroscopic dot upon a larger hydrophobic (water repelling) field. Contact of the surface with an aqueous solution will result in individual drops of water that adhere to the dots but not to the space between the dots. Each dot forms a well.

The term “optical contact” between a first part and a second part means that the two parts are positioned in direct contact or separated by a space such that light leaving one part (after reflection or passing through that part) and subsequently enters or reflects from the surface of the other part. In some cases optical contact is facilitated by physical contact between the surfaces of the two parts. An (refractive) index matching fluid, gel or soft material or paste may be interposed between the parts to fill any gaps between them and limit reflective loss. The index matching substance is transparent to the radiation used and has an index of refraction that is matched to the optical components.

The term “prismatic” means to bend light used in an optical measurement with respect to the surface of a target transparent medium such that the light enters the surface at an angle closer to the perpendicular of the target surface. A light transparent prism may be used in a prismatic fashion by choosing suitable angles and placement of the prism near to or in contact with the target.

The term “prismatically direct” with respect to a transparent optical structure means that the optical structure alters the path of light that passes through it by virtue of one or more controlled surface angles. As well appreciated by a skilled artisan, prisms are used to both split broadband light into different frequencies, combine broadband light into narrower bandwidths, and alter the direction of light passing through them.

The term “prismatic feature” means an optical feature whereby light passes through two surfaces of one or more optical structures in a manner that prismatically directs the light.

The term “internal reflection element” means a transparent optical structure such as a microstructure of an etched semiconductor chip, a protruding light pipe or channel into a sample well or a surface in contact with a sample that undergoes total internal reflection at the interface where contact with the sample occurs.

Other combinations of the inventive features described herein, of course can be easily determined by a skilled artisan after having read this specification, and are included in the spirit and scope of the claimed invention. The references cited above are specifically incorporated in their entireties by reference and represent art known to the skilled artisan. Priority documents U.S. 60/428,241 filed Nov. 22, 2002 and U.S. Ser. No. 10/366,464 filed Feb. 14, 2003 are specifically incorporated by reference.

1. An apparatus for conducting an analysis of a biological sample, comprising:
a sphere designed for application to the biological sample, the sphere being suited for receiving infrared radiation that can be communicated to the biological sample and reflected from the biological sample, the communication to and from the biological sample being through the sphere when the infrared radiation is received.

2. An apparatus for conducting an analysis of a biological sample as recited in claim 1, further comprising:
a conduit configured to hold the sphere at a first end of the conduit, the conduit capable of propagating the infrared radiation from a radiation source at a second end of the conduit to the sphere and capable of propagating the infrared radiation reflected from the biological sample and received by the sphere to the second end of the conduit.

3. An apparatus for conducting an analysis of a biological sample as recited in claim 1, wherein the sphere has an index of refraction greater than a surrounding solution containing the biological sample.

4. An apparatus for conducting an analysis of a biological sample as recited in claim 1, wherein the sphere is configured to be covered by a biological capture layer.

5. An apparatus for conducting an analysis of a biological sample as recited in claim 1, wherein the biological capture layer is one of an antibody or an analyte in solution.

6. An apparatus for conducting an analysis of a biological sample as recited in claim 1, wherein the communication through the sphere is configured to conduct internal reflection sampling.

7. An apparatus for conducting an analysis of a biological sample as recited in claim 2, wherein the conduit is infrared transparent.

8. An apparatus for conducting an analysis of a biological sample as recited in claim 2, wherein the conduit is one of a tube or a fiber.

9. An apparatus for conducting an analysis of a biological sample as recited in claim 2, wherein a cross-sectional dimension of the conduit controls a range of infrared wavelengths that propagates through the conduit.

10. An apparatus for conducting an analysis of a biological sample as recited in claim 1, wherein the infrared radiation is directed out of the sphere allowing internal reflection analysis and the reflected infrared radiation is capable of entering back into the sphere.

11. An apparatus for conducting an analysis of a biological sample as recited in claim 1, wherein the sphere made from an infrared transparent medium.

12. A method to analyze a biological sample with a sphere, comprising:
applying a sphere to a solution including the biological sample;
attracting the biological sample to the sphere;
attaching the sphere to a conduit;
applying infrared radiation through the conduit and the sphere to the biological sample; and
transmitting infrared radiation reflected back from the biological sample through the sphere and the conduit to an infrared radiation detector.

13. A method to analyze a biological sample with a sphere as recited in claim 12, wherein the sphere is an infrared transparent medium.

14. A method to analyze a biological sample with a sphere as recited in claim 12, wherein attracting the biological sample to the sphere includes attracting the biological sample to one of an antibody or an analyte in solution on a surface of the sphere.

15. A method to analyze a biological sample with a sphere as recited in claim 12, further comprising:
analyzing the infrared radiation reflected back to the biological sample with the infrared radiation detector.

16. A method to analyze a biological sample with a sphere as recited in claim 12, wherein applying an infrared radiation through the conduit and the sphere to the biological sample includes conducting internal reflection sampling with the sphere.

17. A method to analyze a biological sample with a sphere as recited in claim 12, wherein applying the sphere to a solution includes covering the sphere with a biological capture layer.

18. An apparatus for conducting an analysis of a biological sample, the apparatus comprising:
a plurality of fibers configured to collect spectral information from multiple biological samples;
infrared optics coupled to the plurality of fibers, the infrared optics being configured to pass light into the plurality of fibers and receive light from the plurality of fibers; and
a detection system configured to simultaneously measure the light received from the plurality of fibers.

19. A system for conducting an analysis of a biological sample as recited in claim 18, wherein the plurality of fibers is coated with one of an antibody or an analyte in solution capable of attracting the biological sample.

20. A system for conducting an analysis of a biological sample as recited in claim 18, wherein the plurality of fibers is an infrared radiation transparent medium.

21. A system for conducting an analysis of a biological sample as recited in claim 18, wherein a light beam transmitted through the plurality of fibers undergoes multiple internal reflections which are resolved by hyperspectral imaging.

22. A method to analyze a biological sample with a sphere, comprising:
applying the biological sample to the sphere;
applying infrared radiation through the sphere and to the biological sample; and
receiving reflected infrared radiation from the sphere for analysis of the biological sample.

23. A method to analyze a biological sample with a sphere as recited in claim 22, wherein the sphere is an infrared transparent medium.

24. A method to analyze a biological sample with a sphere as recited in claim 22, wherein applying the biological sample to the sphere includes attracting the biological sample to one of an antibody or an analyte in solution on a surface of the sphere.

25. An apparatus for conducting an analysis of a biological sample, comprising:
an object designed for application to the biological sample, the object being suited for receiving infrared radiation that can be communicated to the biological sample and reflected from the biological sample, the communication to and from the biological sample being through the object when the infrared radiation is received.

26. An apparatus for conducting an analysis of a biological sample as recited in claim 25, wherein the object is one of a sphere or a fiber.

27. An apparatus for conducting an analysis of a biological sample as recited in claim 25, wherein the object has an index of refraction greater than a surrounding solution containing the biological sample.
